(19) United States
(12) Patent Application Publication
(10) Pub. No.: US 2018/0133110 A1
(43) Pub. Date: May 17, 2018

(54) MEDICATION DISPENSING AND MONITORING DEVICE AND SYSTEM AND METHODS OF FORMATION AND USE

(71) Applicant: Robert Bickley, Paradise Valley, AZ (US)

(72) Inventor: Robert Bickley, Paradise Valley, AZ (US)

(21) Appl. No.: 15/666,469

(22) Filed: Aug. 1, 2017

Related U.S. Application Data
(60) Provisional application No. 62/369,211, filed on Aug. 1, 2016.

(51) Int. Cl. A61J 7/04; A61J 7/00; G16H 20/13

(52) U.S. Cl. CPC A61J 7/0481; A61J 7/0069; A61J 7/0084; A61J 7/0418; A61J 7/0454; G16H 40/67; A61J 2200/74; A61J 2205/70; A61J 2205/10; A61J 2200/30; G16H 20/13

(57) ABSTRACT

A medication dispensing and compliance monitoring system is described. The system includes a dispenser unit which holds a tray containing an array of sequentially ordered medication canisters and a mobile computing device physically incorporated into the dispenser unit. The mobile computing device, such as a cell phone, acts as a communications link and contains processing, memory, and user interface capabilities of the system. Each medication canister is dispensed by the system at a prescribed time, and contains a single dose of one or more medications. Sensors within the dispenser create data documents times wherein medications are dispensed, which can be uplinked to remote parties, such as healthcare providers, caretakers, and family members. Coupling with patient physiologic monitoring devices is also provided. A novel loading tray for accurately loading a plurality of prescribed medications into each medication canister is also disclosed.

### CROSS REFERENCE TO RELATED APPLICATION

This application claims priority from U.S. Patent Application No. 62/369,211 to Robert Bickley, filed Aug. 1, 2016 and entitled “Method for Medication Compliance and Patient Monitoring with Minimal Patient Interaction,” the disclosures of which are hereby incorporated entirely herein by reference.

### BACKGROUND OF THE INVENTION

### Technical Field

The present invention relates generally to systems of devices and methods for dispensing medications. Specifically, the present invention relates to a medication dispensing and monitoring system, including methods of formation and use.

### State of the Art

Compliance with a schedule of medications is difficult for many people. Serious medical conditions, cognitive decline common with advancing age, and the complexity of medication regimens create a situation wherein taking the correct dose of the prescribed medication at the proper time of day may be impossible. Additionally, some pills are small and difficult to see and medication labels having prescribing instructions may be difficult to read. Pill vials are easily spilled, and pills lost on the floor may be eaten by pets or small children.

An additional problem is securing large quantities of potentially dangerous medications from inadvertent consumption by children or the patient, from theft, or from tampering.

What is needed, therefore, is a device and method of use to dispense the correct prescribed dose of a medication to a patient which can secure a plurality of medications against theft or tampering, which can hold a week or a full month's worth of medications, is inexpensive to manufacture, and which can be easily operated by a person with limited cognitive abilities.

### DISCLOSURE OF THE INVENTION

The present invention relates generally to systems of devices and methods for dispensing medications. Specifically, the present invention relates to a medication dispensing and monitoring system, including methods of formation and use.

Disclosed is a medication dispensing and monitoring system comprising a dispenser having a container tray having a dispensing tray located proximate to a first end; an actuator coupled to a sensor, a plurality of medication canisters, wherein each medication canister is removably contained in the container tray, an actuator operatively coupled to the container tray, wherein energizing the actuator causes a transfer of the medication canister from the container tray into the dispensing tray; and a communications link having a first microprocessor coupled to the actuator and the sensor, a mobile computing device having a second microprocessor, a wireless link to a cellular network, a display coupled to the second microprocessor, a user interface coupled to the second microprocessor, a memory coupled to the second microprocessor, a prescribed dosing schedule residing on the memory, and an application residing on the memory, wherein the actuator transfers the medication canister from the container tray into the dispensing tray at a prescribed time in response to a signal received from the microprocessor at a time according to the prescribed schedule.

In some embodiments, the sensor is a pressure sensor. In some embodiments, the sensor if a position sensor. In some embodiments, the sensor is an optical sensor.

In some embodiments, the system further comprises a microphone and a speaker communicatively coupled to the second microprocessor. In some embodiments, the system further comprises a camera communicatively coupled to the second microprocessor.

In some embodiments, one of the plurality of medication canisters comprises a unitary body with at least two sides; and a lid mechanically coupled to the unitary body, wherein the lid is movable between a closed position and an open position with respect to the unitary body. In some embodiments, the communications link comprises a wireless coupling between the memory and a health monitoring system.

In some embodiments, the system further comprises an emergency button coupled to the communications link. In some embodiments, the communications link comprises a data upload to a healthcare provider, a family member, or an emergency service provider.

In some embodiments, the system further comprises a loading tray removably coupled to the container tray, having an array of compartments comprising a pill corral, a pill chute coupled to the pill corral by a first ramp, a loading area coupled to the pill corral by a second ramp, and a fill port coupled to the pill corral by a gate movable between a gate closed position and a gate open position; a sliding cover slideably coupled to the loading area and interposed between the loading area and the fill port; and the medication canister located beneath the sliding cover in the container tray.

In some embodiments, the loading tray additionally comprises a magnifying viewer.

In some embodiments, the system further comprises a camera communicatively coupled to the memory and the second microprocessor; and a prescription container platform, wherein the camera is activated to capture an image of a prescription label affixed to a medication container placed on the prescription container platform, and transfer the image to the memory.

The medication dispensing and monitoring system of claim 12, wherein the image is a panoramic image.

In some embodiments, the communications link comprises a receiving dock and a mobile computing device coupled to the receiving dock, wherein the mobile computing device is electrically coupled to the first microprocessor, and comprises the second microprocessor and the memory.

In some embodiments, the actuator is prevented from functioning in response to a signal from the first microprocessor under a condition wherein the mobile computing device becomes electrically uncoupled from the actuator.

Disclosed is a medication dispensing and monitoring system comprising a power source; a dispenser having a plurality of medication canisters secured within a locked container tray, a dispensing tray external to the locked container tray, an actuator electrically coupled to the power source, and a first microprocessor coupled to the actuator; a mobile computing device electrically coupled to the power source, the mobile computing device having a second microprocessor, a memory with an application and medication schedule resident on the memory, wherein the power source energizes the actuator in response to a signal from the second microprocessor to transfer one of the plurality of medication canisters into the dispensing tray at a time indicated by the medication schedule; wherein the mobile computing device is fixedly coupled to the dispenser and wherein removal of the mobile computing device enables the first microprocessor to cause activation of the actuator at the time indicated by the medication schedule.

In some embodiments, the first microprocessor causes a signal to be transmitted to a healthcare provider in response to a condition wherein the remote computing device becomes removed from the dispenser, or wherein the remote computing device becomes electrically uncoupled from the actuator.

Disclosed is a method of using a medication dispensing and monitoring system comprising steps coupling a loading tray to a container tray of the medication dispensing and monitoring system having a dispenser holding plurality of medication canisters; loading the plurality of medication canisters with a medication; removing the loading tray and inserting the container tray having the plurality of loaded medication canisters into a dispenser; activating a mobile computing device coupled to the dispenser, wherein the mobile computing device comprises a medication prescription resident on a memory of the mobile computing device; and dispensing a single medication canister at a prescribed time according to a dose time instruction comprised by the medication prescription.

The foregoing and other features and advantages of the present invention will be apparent to those of ordinary skill in the art from the following more particular description of the invention and its embodiments, and as illustrated in the accompanying drawing figures.

### BRIEF DESCRIPTION OF THE DRAWINGS

FIG. 1 is a perspective view of a medication dispenser and monitoring system;

FIG. 2 is a perspective view of a loading tray coupled to a container tray of a medication dispenser and monitoring system;

FIG. 3 is a perspective view of a container tray of a medication dispenser and monitoring system holding a plurality of medication canisters;

FIG. 4 is a perspective view of a pill caddy;

FIG. 5 is a perspective view of a phone dock of a medication dispenser and monitoring system coupled to a cellular phone;

FIG. 6 a top view of a phone dock of a medication dispenser and monitoring system coupled to a cellular phone;

FIG. 7 is a side view of a phone dock of a medication dispenser and monitoring system coupled to a cellular phone;

FIG. 8 is a side view of a phone dock of a medication dispenser and monitoring system coupled to a cellular phone in a closed position;

FIG. 9 a side view of a phone dock of a medication dispenser and monitoring system coupled to a cellular phone in an open position;

FIGS. 10a-c are views of a medication canister of a medication dispenser and monitoring system;

FIG. 11a is an end view of a loading tray of a medication dispenser and monitoring system;

FIG. 11b is a top view of a medication row of a loading tray of a medication dispenser and monitoring system;

FIG. 11c is a perspective view of prescription containers of a medication dispenser and monitoring system;

FIG. 12 is a side view of a loading tray of a medication dispenser and monitoring system;

FIGS. 13a-c are perspective views of a loading tray coupled to a pill caddy of a medication dispenser and monitoring system;

FIG. 14a is a perspective view of a mobile computing device of medication dispenser and monitoring system configured as a pill magnifier;

FIG. 14b is a perspective view of a medication dispenser and monitoring system with a camera stage;

FIG. 15a-d are side cutaway views of a container tray being loaded into a dispenser to engage with an actuator of a medication dispenser and monitoring system;

FIG. 16a-f are side cutaway views of a dispenser of a medication dispenser and monitoring system throughout a dispensing cycle;

FIGS. 17a-d are detail side-cutaway views of an actuator mechanism of a medication dispenser and monitoring system;

FIGS. 18a-b are side cutaway views of a dispenser of a medication dispenser and monitoring system having an internal latch;

FIG. 19 is a side cutaway view of a dispenser of a medication dispenser and monitoring system;

FIG. 20 is a circuit block diagram of a medication dispenser and monitoring system;

FIG. 21 is a schematic representation of a medication dispenser and monitoring system;

FIG. 22 is a schematic representation of a method of using a medication dispensing and monitoring system;

FIG. 23 is a perspective view of a multiple pill dispenser installation of a medication dispensing and monitoring system; and

FIG. 24 is a perspective view of a healthcare facility medication dispensing center of a medication dispensing and monitoring system.

### DETAILED DESCRIPTION OF EMBODIMENTS OF THE INVENTION

As noted herein, the present disclosure relates generally to systems of devices and methods for dispensing medications. Specifically, the present invention relates to a medication dispensing and monitoring system, including methods of formation and use.

Embodiments of the disclosed invention organize, control, and monitor dosing of medications, and may include remote physiologic monitoring. The medication dispensing and compliance monitory system may help enable treatment of seriously ill patients in a variety of settings, whether at home, in the hospital, or in an extended care or assisted living facility. The invention includes a locked medication dispenser which is readily portable for travel. Use of the medication dispensing and compliance monitoring system additionally assist healthcare providers in tracking medication compliance, assuring that the patient can easily adhere to a prescribed schedule of medications and participate in physiologic monitoring, regardless of location and circumstance. Use of the system should help to reduce the frequency of medication errors, particularly in an outpatient setting.

If the patient should miss a scheduled medication time, some embodiments of the medication dispensing and compliance monitoring system communicate a third-party alert, wherein designated stakeholders of the patient's family and healthcare team can monitor compliance. In some embodiments, the system can establish audiovisual communication between the patient and stakeholders, including emergency response personnel, for immediate remedial action.

Currently, a patient or a member of the patient's family often use a simple pill box to aid in compliance with a prescribed medication regimen, filling the box with doses of the various prescribed medications. This can be a tedious and error-prone task if the patient's medication regimen includes multiple drugs on different dosing schedules.

It is desirable for the frequency of medication errors be minimized or eliminated completely. Medication errors include taking the wrong medication or the wrong dose of the correct medication, for example, from duplication or omission. Errors also include taking the correct dose of medication at an incorrect time, including late or missed doses. Frequent manipulation of pill containers may result in accidental spills wherein medication pills are lost or contaminated. Some medications comprise “sticky pills,” such as gel caps, which can be retained in a conventional pill box, remaining unnoticed by or inaccessible to the patient.

Patients at home often receive prescribed medications from a retail pharmacy, either locally or through the mail. Some embodiments of the medication dispensing and compliance monitoring system may receive and store information comprised by the prescription label of each dispensed medication container, such that a dosing schedule may be compiled and stored by the system.

The medication dispensing and compliance monitoring system may also be used in a hospital, extended care, or assisted-living facility to ensure compliance with the prescribed medication regimen. During hospitalization, the patient's prescription regimen may be frequently changed, according to the patient's condition. Whereas an initial schedule of prescribed doses is provided by the hospital pharmacy, nursing staff sometimes may be relied upon to make ad hoc changes, pursuant to a treating provider's orders. Such revisions are facilitated by use of certain embodiments of the medication dispensing and compliance monitoring system at the nursing station, or in the patient's room. Each time medications are changed or added it is important to check for unintended drug interactions and to ensure that each new dose contains the correct assortment of pills. Some embodiments of the instant invention can be used in a multiple unit configuration that is suitable for a healthcare facility medication dispenser as would be utilized on a hospital floor.

Often medication requirements include the use of pain relief medications such as opioids on an “as required” basis. Embodiments of the disclosed invention may manage “as-required” dosing in at least two ways: 1) a maximum allowed frequency of “as required” doses is set to prevent overmedication; and 2) the as-needed medication is stored and dispensed in a secure manner that hampers attempts at tampering or theft. Additionally, embodiments of the invention may create a stored electronic record to document the number and frequency of dispensed doses.

A highly important element of the system is an embedded mobile computing device, such as a cell phone, a smartphone, or a tablet device. Other elements comprised by some embodiments of the invention include a recycled cellular phone to provide a display, user interface for data entry, microprocessor, and a memory at low-cost. The embedded mobile computing device also acts as a 2-way data communications link and may comprise Bluetooth, Wi-Fi, and cellular network connectivity. A clock, back-up battery power source, software application for monitoring medication dose schedules, and data collection and storage are also provided by the embedded mobile computing device, in some embodiments.

As technology relating to mobile computing devices changes over time, the medication dispensing and compliance monitoring system is easily upgraded by upgrading the embedded mobile computing device to a newer-generation device.

A removable container tray is secured within the dispenser and holds and maintains medication canisters in dose-sequential order. The container tray may also be locked into the pill dispenser and can only be removed by entering a code or unlocking with a key. The removable container tray makes it easy to reload several days of medication doses into the pill dispenser.

An actuator assembly is a control element of the pill dispenser that releases or “vends” medication canisters. This electromechanical device includes a modest amount of circuitry to monitor critical functions, communicate with the embedded mobile computing device, and drive one or more actuator motors upon receipt of a vend command.

In some embodiments (not shown in the drawing figures), installation of multiple medication dispensing and compliance monitoring systems is provided for use by a healthcare facility responsible for patient medications, such as a hospital floor, an extended care facility, or an assisted living facility, for example. With use by multiple patients in a healthcare facility, a single control panel may be used to service and monitor all the medication dispensers in a rack of dispensers. Each medication dispenser may have its own IP address and interface with the control panel using a wired or wireless communications link. A non-limiting example of operation is on a hospital floor. When it is time for a patient to receive a dose of a medication(s), a display on an embedded mobile computing device shows the time of day and a patient location, such as a room number. An indicator light, such as a green LED light, flashes, prompting a caregiver to activate a switch, such as pressing a button on the dispenser, causing the dispenser loaded with medication canisters for that patient to vend a canister containing a single dose of medications. The medication canister may contain a label with identifying information for the medication(s) and the patient, for additional verification by the caregiver. Wherein multiple patients are scheduled to receive medications at the same time, the system can cycle through each patient, advancing in sequence to the next patient and patient location each time the vend switch is activated.

The multiple pill dispenser installation is an important solution for “as required” medications such as pain relief medication. It is important to manage and monitor these medications so that they are not administered too frequently or when not needed.

In one non-limiting example, each pill dispenser tracks all medications dispensed and contains four columns of seven pill containers per column, the pill dispenser can serve four patients with seven doses available for each patient. In an additional non-limiting example, multiple pill dispenser installation are six wide and four deep such that 20 patients have an individual pill dispenser and 16 patients have available seven doses of “as required” (p.r.n.) medications available before a refilling of the dispenser is necessary.

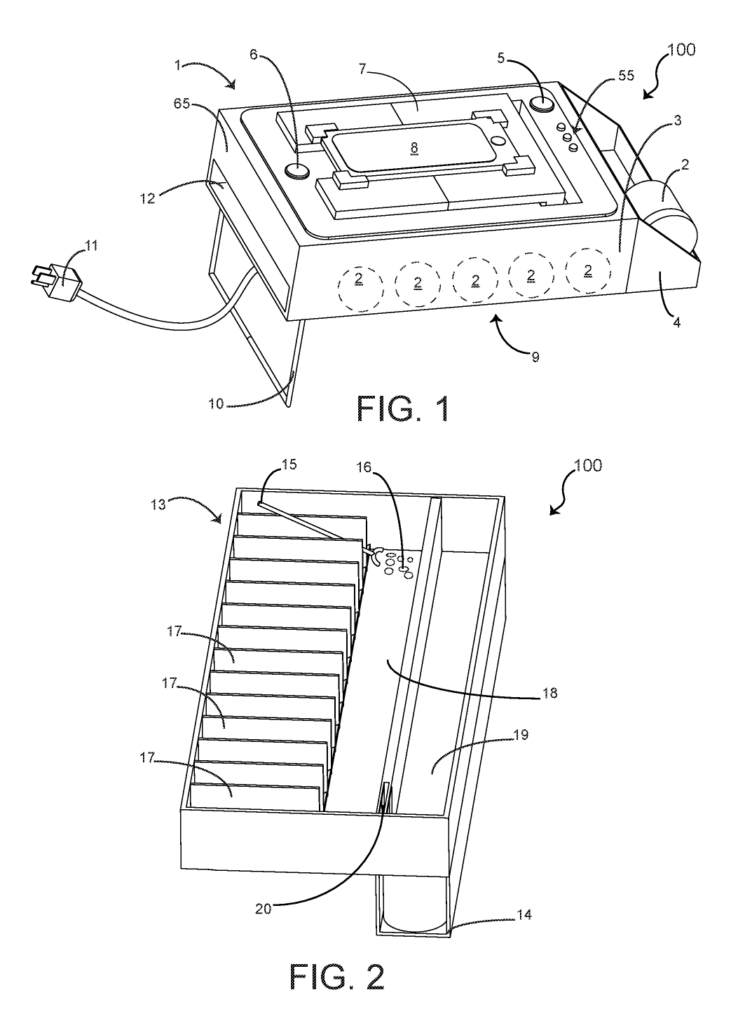

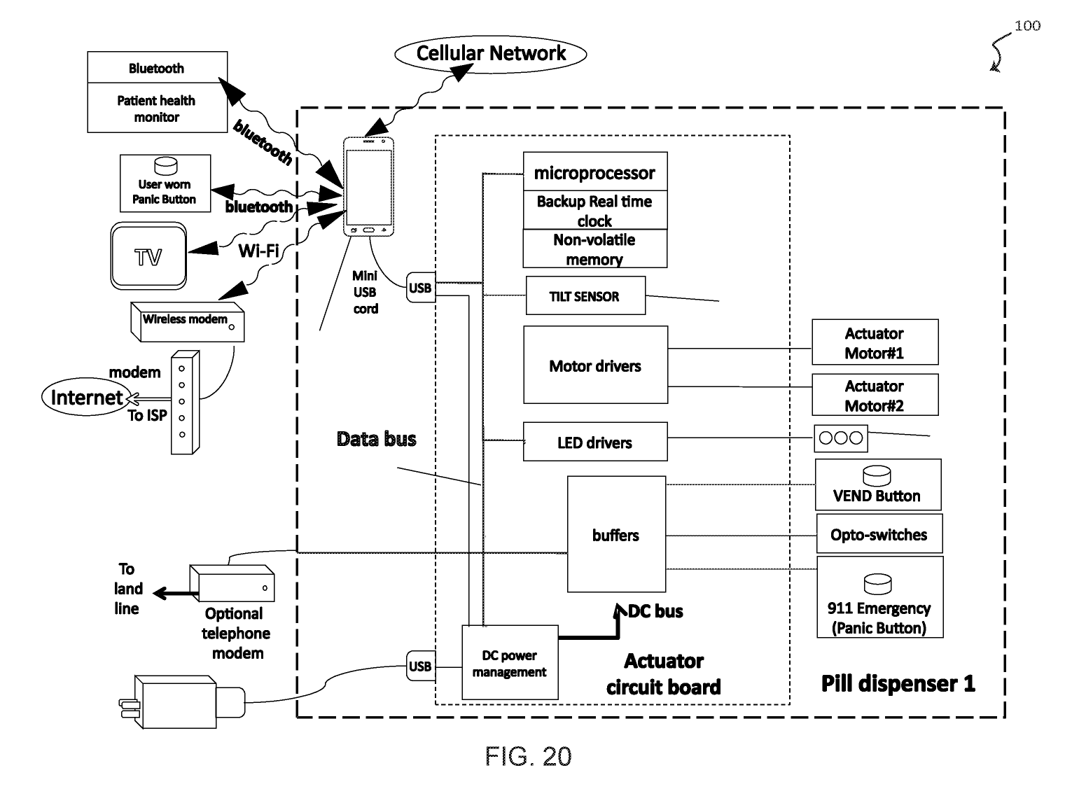

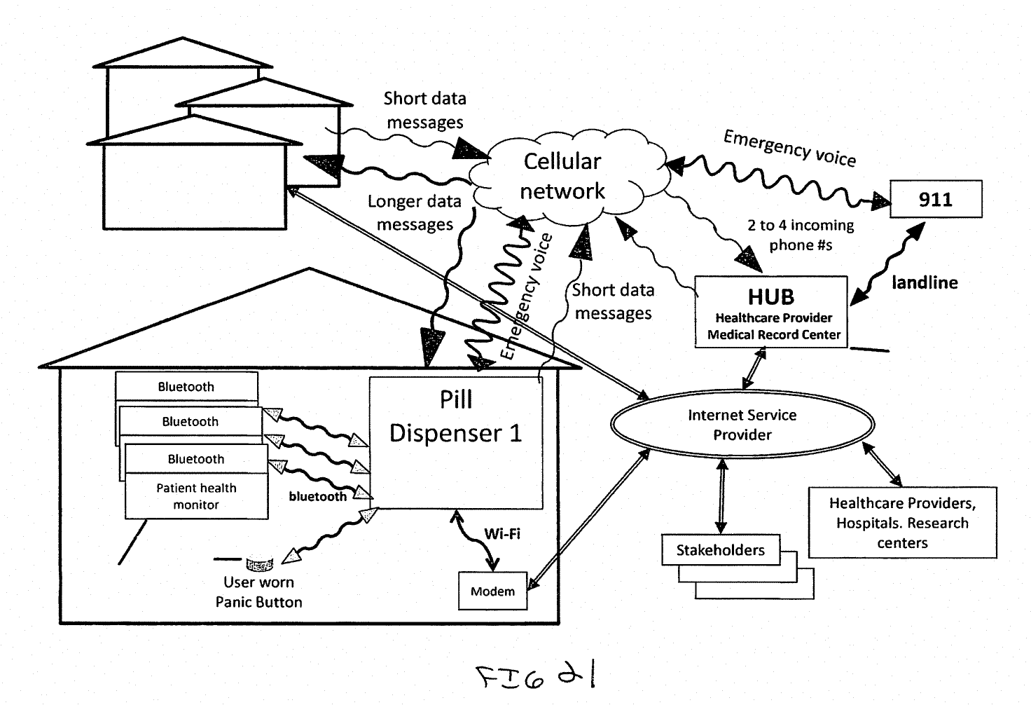

FIG. 1 is a perspective view of a medication dispenser and monitoring system. FIG. 1 shows a medication dispenser and monitoring system 100. System 100, generally speaking, has three primary components—a dispenser 1, a loading tray 13 (See FIG. 2), and a communications link 35 (See FIG. 20). Dispenser 1, as shown in FIG. 1, and in some other embodiments, comprises a housing 65 holding a container tray 12 and an actuator 3.

Container tray 12 is loaded with a plurality of medication canisters 2 in an ordered canister array 9. A loaded container tray 12 is inserted into dispenser 1 within housing 65 prior to use of system 100. In some embodiments, a tilt stand 10 is coupled to dispenser 1 after insertion of container tray 12, locking container tray 12 into position within housing 65 of dispenser 1.

Once loaded into dispenser 1, container tray 12 may be securely locked into dispenser 1 by tilt stand 10. Insertion of removable tilt stand 10 activates an internal latch which engages with a connecting rod of tilt stand 10, locking container tray 12 in place. In some embodiments, an electronic combination lock is operatively coupled to the internal latch, wherein the internal latch can only be released by entering a release code on a user interface of mobile computing device 8. In some embodiments, a conventional physical key is inserted into a lock disposed on housing 65 of dispenser 1 to release the internal latch.

Tilt stand 10 places dispenser 1 at an incline, when installed (coupled) to dispenser 1. In some embodiments, the incline is about seven (7) degrees. In some embodiments, the incline is between about five (4) and about ten (10) degrees. In some embodiments, the incline is greater than about ten (10) degrees. Loading tray 13 provides a device for accurately filling each medication canister 2 with a single dose of one or more medications and is shown in later drawing figures and discussed in detail herein below.

Dispenser 1 additionally comprises an actuator assembly with an actuator 3. Actuator 3, in some embodiments, is an electrically activated mechanical device which 1) locks medication canisters 2 in place within container tray 12; and 2) when activated, transfers a single medication canister 2 out of container tray 12 for retrieval by the user from dispensing tray 4. Also shown by subsequent drawing figures and discussed in detail herein below is communications link 35.

The ordered canister array 9 is configured such that dispenser 1 vends single medication canisters 2 on the day and at the time of day prescribed by the user's health care provider. A mechanism having at least one actuator 3 locks the lead medication canister 2 in place, preventing removal of any medication canister 2 from container tray 12. At the prescribed time, or, in some embodiments, upon receipt of a signal from a user-activated switch, actuator 3 vends a single medication canister 2 by causing canister 2 to move from container tray 12 into a dispensing tray 4, wherefrom the user can remove canister 2 from dispensing tray 4 and retrieve her medications from an interior of canister 2. As also shown by FIG. 1, dispenser 1 additionally comprises a vend switch 5, an emergency switch 6, and a phone dock 7 coupled to a mobile computing device 8, in some embodiments. Electrical power to energize actuator 3 comes from a power source, which may be a battery, a cell-phone battery, or an auxiliary power adaptor 11. A tilt stand 10, coupled to dispenser 1 may be fixed in place or moveable, and placed dispenser 1 on an incline wherein medication canister 2, once moved by actuator 3, rolls by gravity into dispensing tray 4, where it may be retrieved by the user of system 100. After actuator 3 moves medication canister 2 out of container tray 12, the medication canister(s) 2 that remain in container tray 12 advance by gravity until the next medication canister 2 sequenced in array 9 moves to engage with actuator 3, to be dispensed in the next activation cycle of actuator 3. A tilt stand 10 is removably coupled to dispenser 1, wherein attaching tilt stand 10 to dispenser 1 placed dispenser 1 at an angle, facilitating the transfer of medication canister 2 into dispensing tray 4. Additionally, insertion of tilt stand 10 into dispenser 1 blocks removal of container tray 12 from dispenser 1, in some embodiments, locking container tray 12 in place within a housing of dispenser 1.

Each medication canister 2 holds one dose of a medication. Canister 2 may hold one, or, most commonly, more than one different medication. In some cases, canister 2 may hold three, four, seven, or more different pills, each pill representing a dose of a medication prescribed to be taken at the same time of the day. In this say a patient who needs to take four different medications twice each day will receive a medication canister 2 twice each day, at eight a.m. and eight p.m., for example, each canister 2 holding the four different pills. It will be appreciated, therefore, because some medications might be prescribed once, twice, three, or four times each day, medication canister 2 must be 1) filled with the proper medications; and 2) sequentially ordered in a canister array 9 for vending by dispenser 1 in sequential order and at the proper times of day.

In some embodiments, dispenser 1 also comprises an emergency switch 6, a vend switch 54, and an alert light 55.

FIG. 2 is a perspective view of a loading tray coupled to a container tray of a medication dispenser and monitoring system. With multiple medications prescribed under multiple dosing schedules, the filling and sequential ordering of many days' worth of medication canisters 2 becomes progressively more complicated and subject to error. Accordingly, some embodiments of medication dispenser and monitoring system 100 comprise a loading tray 13, as shown by FIG. 2. Loading tray 13, generally speaking and in some embodiments, is an open rectangular-shaped member divided into multiple compartments. Each compartment comprises a row of two sequential sub-compartments; namely, a pill corral 17, and a pill chute 19. A common, elongated loading area 19 is disposed proximate to one side of loading tray 13, adjacent to a plurality of pill chutes 19. Each aforementioned rows reserved for a single type of medication. A quantity of pills 16 are accumulated as each pill corral 17 and pill chute 18 contribute one or more of each medication. In some embodiments, pills 16 are initially emptied from a medication container (not shown), such as typical vial-type container of pills dispensed by a retail pharmacist pursuant to a prescription. In some embodiments of system 100 wherein canister array 9 comprises a quantity of medication canisters 2 sufficient for an entire month of individual doses, the medication vial may be completely emptied into loading tray 13.

Pills 16 are initially emptied into a pill corral 17, as shown by FIG. 2. After loading pill corral 17, in some embodiments, a user moves a single dose of the medication across a first ramp 26 into a pill chute 18. The prescribed number of pills comprising s single dose of the medication is pushed into pill chute 18. This number may be one, two, or greater than two pills, according to the prescription. In some embodiments, a pill pusher, such as pusher 15, for example, is used to advance the pills across first ramp 26 into pill chute 18. This procedure is then repeated for each medication that is to be taken on the same day at the same time; namely, each medication that will be retrieved from a single medication canister 2. Each medication is initially loaded into pill corral 17 of a new medication row 30 until all of the medications to be dispensed in the single dose from medication canister 2 are loaded into individual pill corrals 17. The dosage number of each separate medication, such as one, two, or greater than two pills, for example, is then moved across each respective first ramp 26 into a corresponding pill chute 18 within each medication row 30. In this way, following transfer of all medications to be contained in a single medication canister 2 into individual pill corrals 17, the user may closely examine loading tray 13 to confirm that the proper number of pills comprising a single prescribed dose of each medication is in its corresponding pill corral 17.

After all of pills 16 in pill corrals 17 have been transferred to pill chutes 18, the user transfers pills 16 from pill chute 18 across a second ramp 27 into a common loading are 19. After all medications comprised by a single dose have been moved into loading area 19, the user may open a gate 20 and transfer pills 16 through a fill port 28, whereupon pills 16 drop into open medication canister 2 positioned directly beneath fill port 28 within pill caddy 14. Pill caddy 14 is moveably coupled to loading tray 13, whereupon after filling of medication canister 2 through fill port 28, pill caddy 14 is advanced such that the next medication canister 2 becomes positioned below and in communication with fill port 28. Filling of each medication canister 2 held by pill caddy 14 continues until all medication canisters 2 within pill caddy 14 are loaded with a dose of medications. Medication canisters 2 are then closed to secure the doses of pills 16.

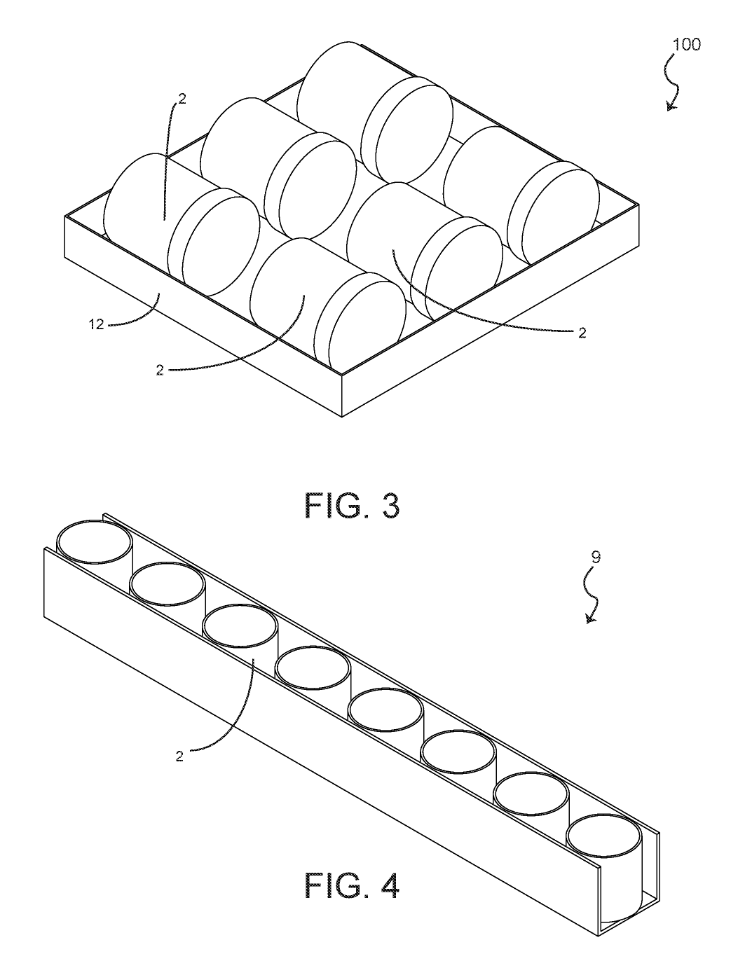

FIG. 3 is a perspective view of a pill caddy. In some embodiments, pill caddy 14 is a generally elongate open tray comprising a shape and dimensions to restrict a row of medication canisters 2 to a generally linear array, such as shown by FIG. 3 Pill caddy 14 functions to maintain the sequence of medication canisters 2 prior to loading medication canisters 2 into container tray 12. FIG. 3 shows an example embodiment of pill caddy 14 to be used in conjunction with loading tray 13 to load seven medication canisters 2. In some embodiments, pill caddy 14 holds less than seven medication canisters 2 and, in some embodiments, greater than seven medication canisters 2.

FIG. 4 is a perspective view of a container tray of a medication dispenser and monitoring system holding a plurality of medication canisters. Container tray 12 is shown by FIG. 4 holding a plurality of medication canisters 2 in a container array 9. Maintaining the plurality of medication canisters 2 in array 9 preserves the order of the plurality of containers 2 such that does of medication are dispensed in the prescribed order by dispenser 1. As shown in the several drawing figures, container tray 12 may hold an array 9 comprising four rows of seven medication canisters 2 in each row for a total of twenty-eight (28) medication canisters 2. After container tray 12 is loaded with the plurality of medication canisters 2, container tray 12 can coupled to dispenser 1 and locked in place, in some embodiments.

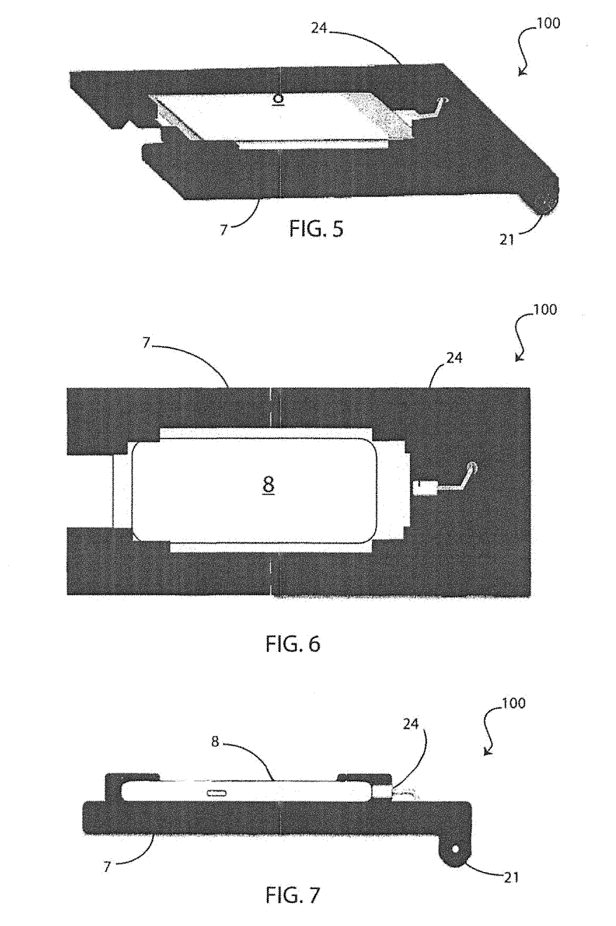

FIG. 5 is a perspective view of a phone dock of a medication dispenser and monitoring system coupled to a cellular phone. FIG. 6 a top view of a phone dock of a medication dispenser and monitoring system coupled to a cellular phone. FIG. 7 is a side view of a phone dock of a medication dispenser and monitoring system coupled to a cellular phone. FIGS. 5-7 show a phone dock 7. Phone dock 7 is designed to couple a mobile computing device, such as a cellular phone or a tablet device, to dispenser 1, wherein components comprised by the mobile computing device operate medication dispenser and monitoring system 100. For example, in some embodiments, a mobile computing device 8 comprises a cell phone having a microprocessor, a display, a user interface comprising a keyboard, and a memory. Phone dock 7, in some embodiments, comprises a plurality of adjustable members, wherein phone dock 7 may be configured to couple a wide range of shapes and sizes of mobile computing device 8, such as different models of cellular telephones, smartphones, and tablet devices from various manufacturers.

In some embodiments, phone dock 7 provides the means to use one of the many commercial low cost re-cycled cell phones as mobile computing device 8 for almost all of the functions in system 100. Because mobile computing device 8 is crucial to the operation of pill dispenser 1, it is mandatory that mobile computing device 8 is securely integrated electrically and mechanically so that it cannot be casually or accidentally removed or stolen.

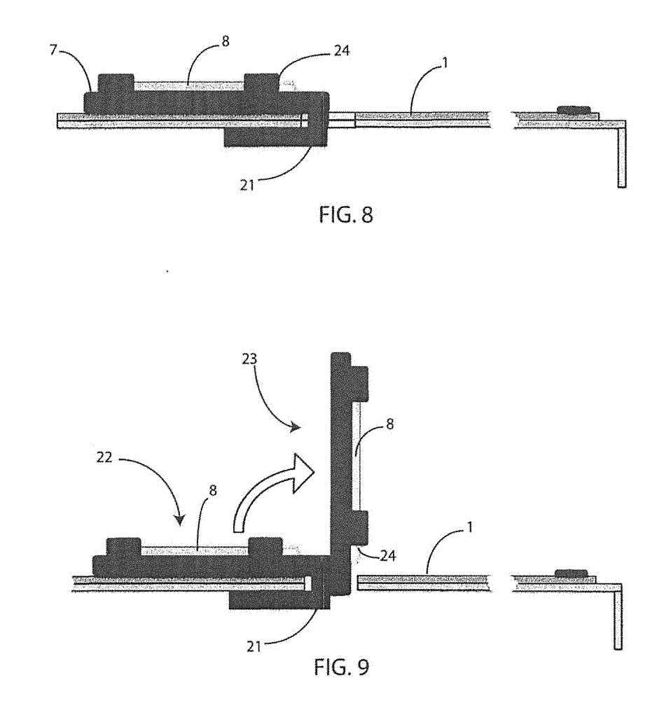

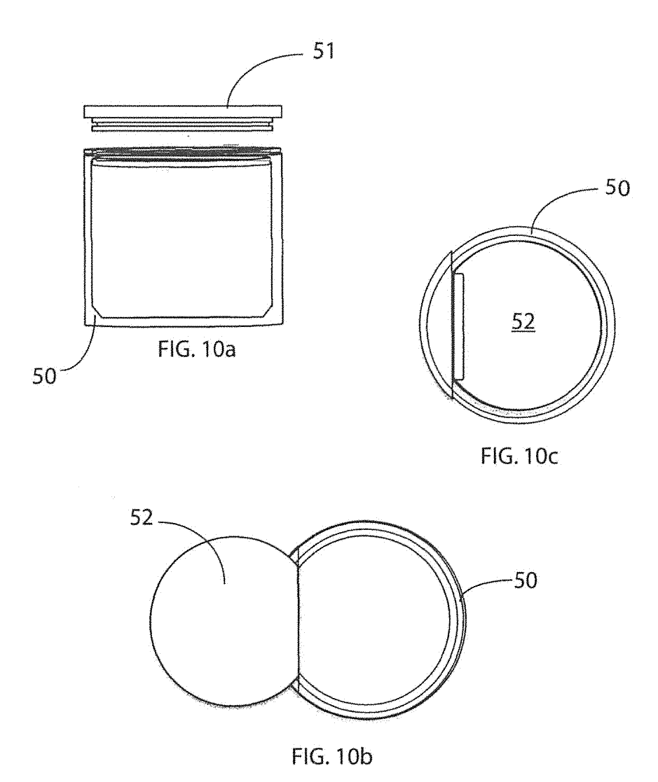

FIG. 8 is a side view of a phone dock of a medication dispenser and monitoring system coupled to a mobile computing device 4 in a closed position. FIG. 9 is a side view of a phone dock of a medication dispenser and monitoring system coupled to a mobile computing device in an open position. As shown by FIG. 9 and FIG. 10, phone dock 7 coupled to mobile computing device 8 may be configured in a closed position 22, as shown by FIG. 8, or an open position 23, as shown by FIG. 9. Transition between closed position 22, open position 23, or any position within a continuum of positions intermediately between closed position 22 and open position 23 is accomplished by rotating phone dock 7 on a hinge 21. In some embodiments of system 100, hinge 21 is a hidden hinge positioned on a cover of dispenser 1 such that hinge 21 is inaccessible to a user of system 100 to resist tampering; i.e., uncoupling and removal of remote computing device 8 from dispenser 1.

FIGS. 10a-c are views of a medication canister of a medication dispenser and monitoring system. FIG. 10a is a side view of one embodiment of medication canister 2 comprising a container 50 and a removable screw-cap 51. FIG. 10b is a top view of an open container 50 and a hinged lid 52 hingedly coupled to container 50. FIG. 10c is a top view of container 50 with hinged lid 52 closed onto container 50. As shown by FIGS. 10a-c, multiple configurations and embodiments of medication canister 2 are possible, including the non-limiting examples shown. Medication canister 2 comprises container 50 and a cover. Container 50 is generally cylindrical, in some embodiments, after removal from container tray 12 by actuator 3, container 50 may roll down the incline of dispenser 1 created by installed tilt stand 10 into dispensing tray 4. The cover is fixedly coupled in some embodiments, such as hinged lid 52, and removably coupled in some other embodiments, such as screw-cap 51. Other configurations of medication canister 2 are within the scope of the disclosed invention, however, each medication canister 2 must comprise a means wherein the container may be opened for the filling or retrieving of medications and securely closed, such that pills or other configured medication dosing forms; i.e., gel caps, blister packs, suppositories, peel packs, ampules, and the like, may be secured therein without spilling, contamination, or loss.

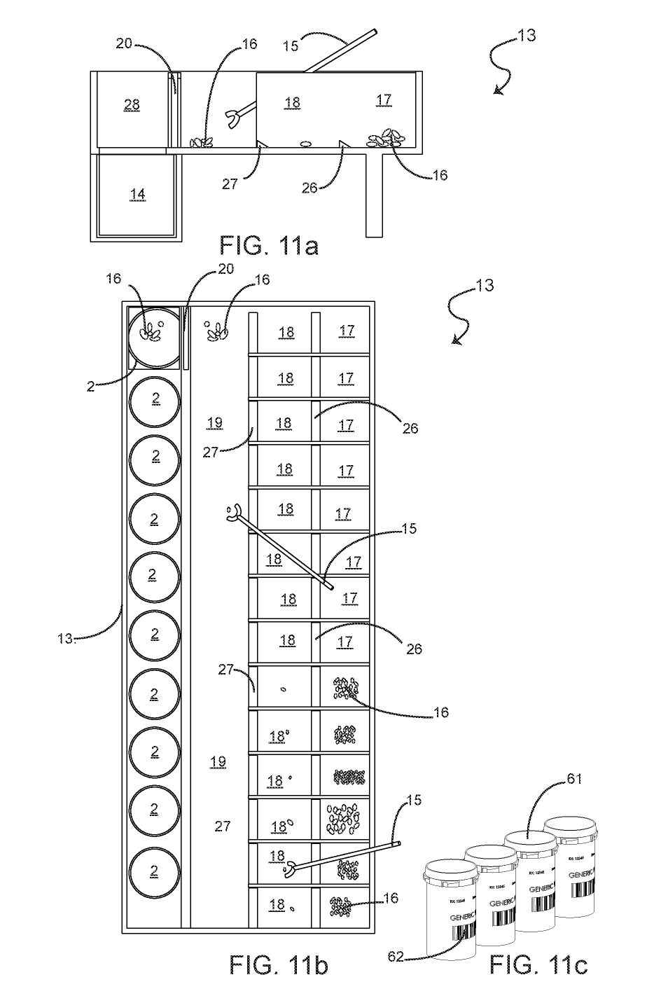

FIG. 11 is a top view of a loading tray of a medication dispenser and monitoring system. In the example embodiment of loading tray 13 shown by FIG. 11, fourteen different medications are accommodated for loading into seven different medication canisters 2, wherein each loaded medication canister 2 comprises a single patients dose of any number and combination of the fourteen (14) different medications to be administered on a particular day at a prescribed time by dispenser 1 of medication dispensing and compliance monitoring system 100.

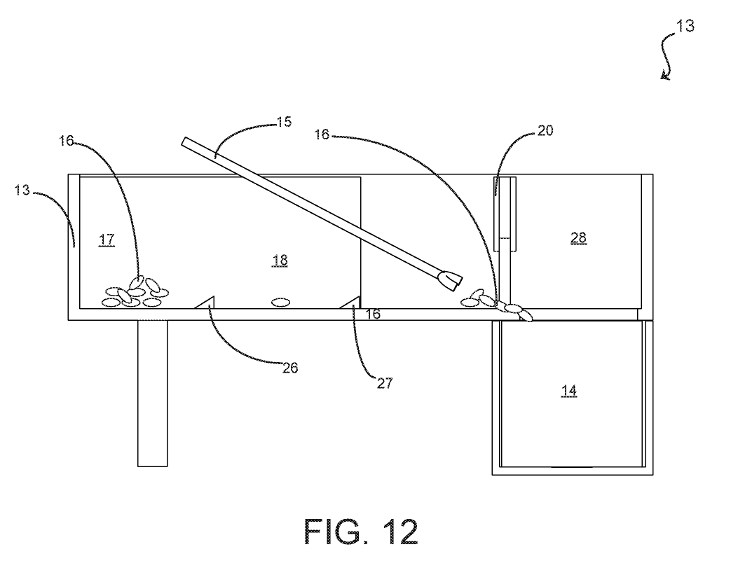

FIG. 12 is a cut-away side view of a loading tray of a medication dispenser and monitoring system. FIG. 12 shows an example medication row 30.

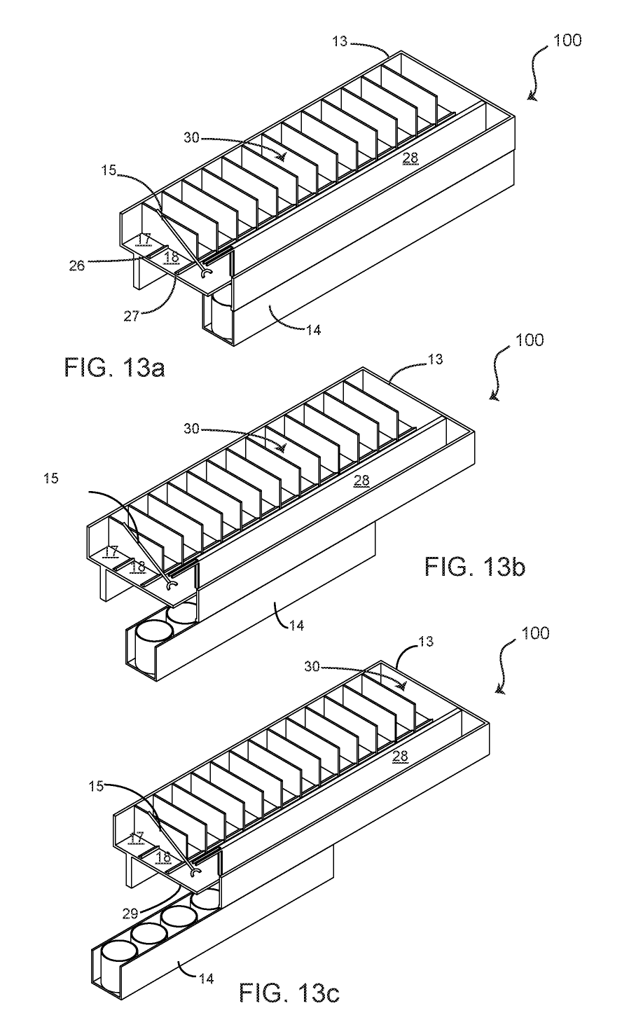

FIGS. 13a-c are perspective views of a loading tray coupled to a pill caddy of a medication dispenser and monitoring system;

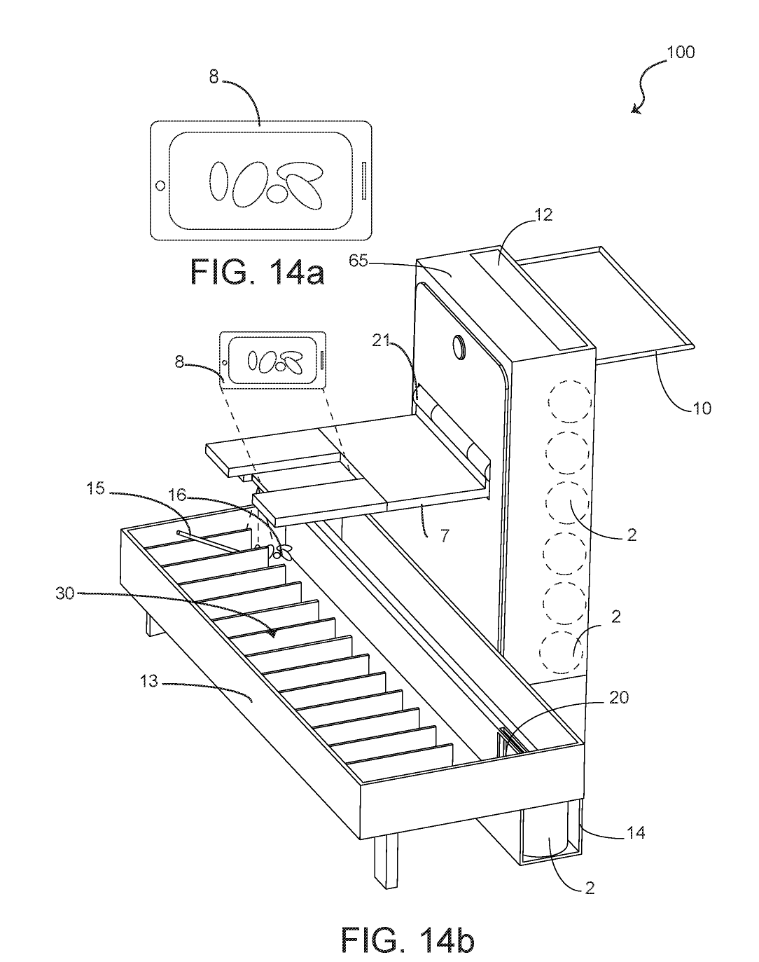

FIG. 14a is a perspective view of a mobile computing device of medication dispenser and monitoring system configured as a pill magnifier. In some embodiments of system 100, dispenser 1 is positioned upright in a position proximate and generally orthogonal to loading tray 13, as shown in FIG. 14a. Phone dock 7 is shown in open position 23, wherein a camera 44 resident on mobile computing device 8 creates a magnified image of pills 16 located in a compartment of loading tray 13.

FIG. 14b is a perspective view of a medication dispenser and monitoring system with a prescription container platform. As shown by FIG. 14b, a prescription container platform 60 is rotatably coupled to a top surface of dispenser 1, wherein a prescription container 61 bearing a medication label 62 is placed on prescription container platform 60 and rotated before an activated camera 44 of mobile computing device 8 to capture an image that is stored on memory 41. In some embodiments, prescription container platform 60 is rotate to image a particular area of medication label 62, such as an area displaying a bar cord, a QSR code, or the like. In some embodiments, prescription container platform 60 rotates prescription container 61 such that camera 44 captures a panoramic image comprising the entirety of medication label 62. In some embodiments, the stored image is retrieved on display 43 as a magnified image, wherein the name of the medication, the prescribed dosing instructions, warnings, and other information displayed on medication label 62 are more easily viewed for reading by a user of medication dispensing and compliance system 100.

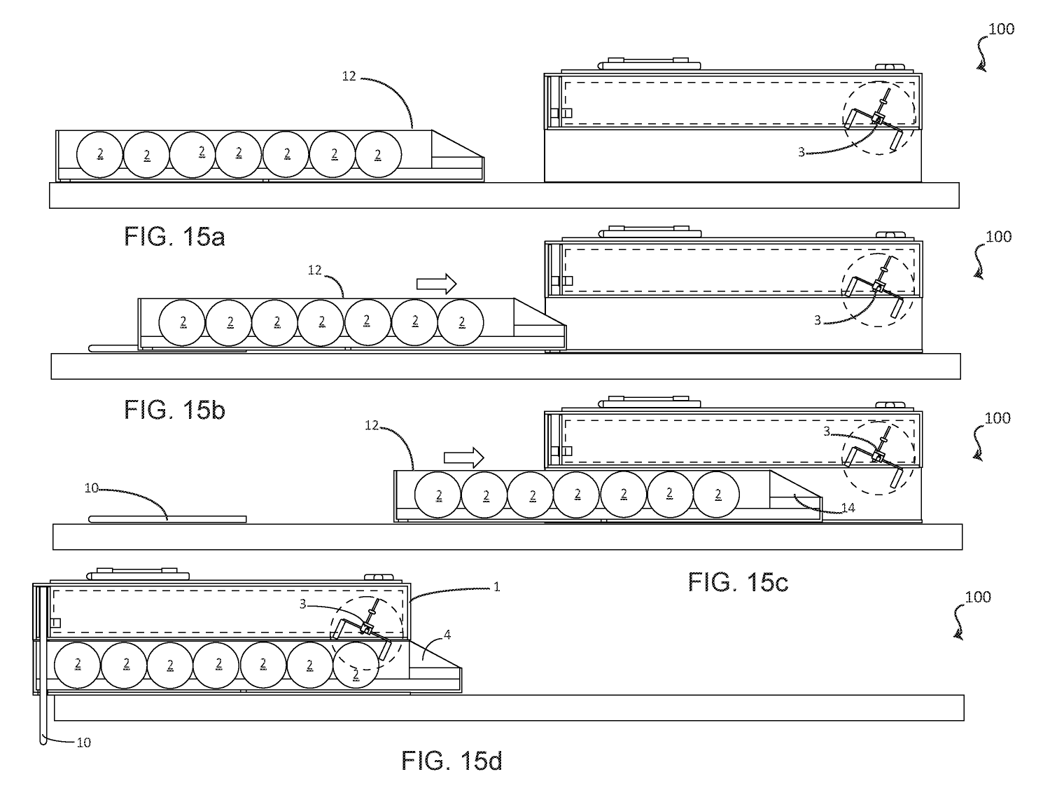

FIGS. 15a-d are side cutaway views of a container tray being loaded into a dispenser to engage with an actuator of a medication dispenser and monitoring system. FIG. 15d shows tilt stand 10 coupled to dispenser land engaging with an internal latch 66 wherein container tray 12 is locked in place within housing 65 of dispenser 1. FIG. 15d additionally shows actuator 3 engaged with a single medication canister 2 after completing full insertion of container tray 12 into dispenser 1, as shown sequentially by FIG. 15a-d.

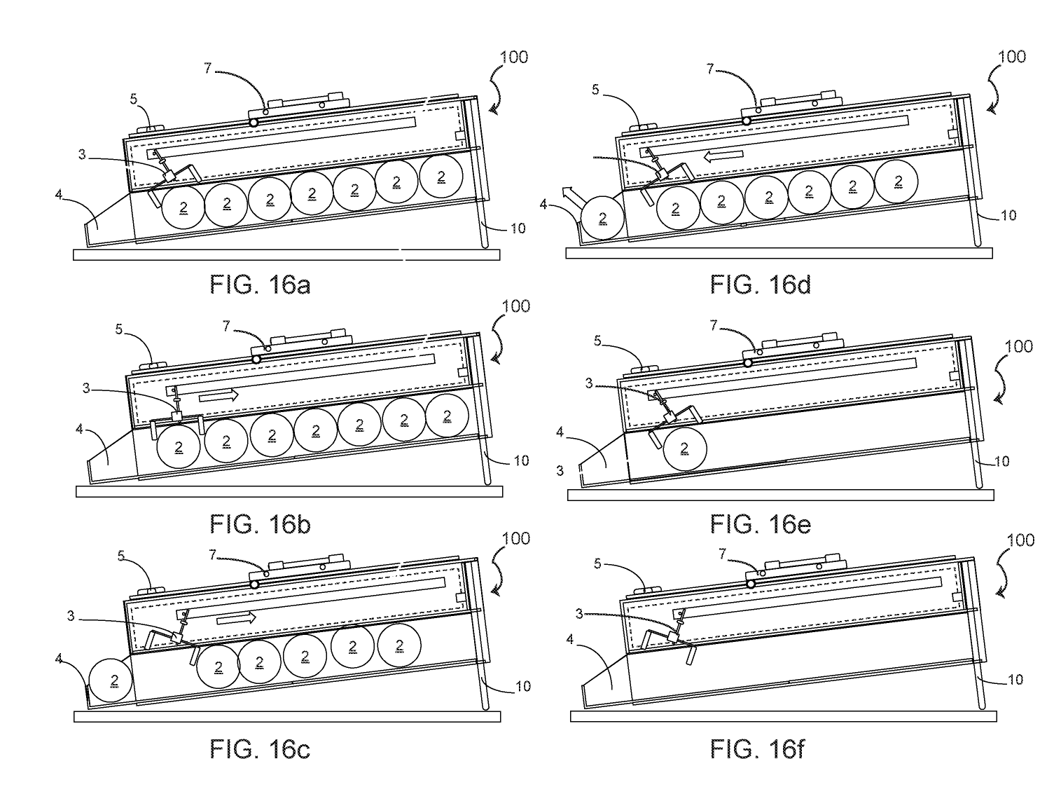

FIGS. 16a-f are side cutaway views of a dispenser of a medication dispenser and monitoring system throughout a dispensing cycle. FIGS. 16a-f show a full dispensing cycle, starting with FIG. 16a a rocker arm of actuator 3 is engaged with medication canister 2. An alert light 55 disposed on housing 65 of dispenser 1 illuminates in response to a signal from mobile computing device 8 at the prescribed time to administer a medication dose to the user of system 100. In some embodiments, a speaker 46 (not shown) generates an audible alert, also in response to receiving a signal from mobile computing device 8. After receiving the alert, the patient or a caregiver responds by activating a vend switch 54, as shown in FIG. 16b. Activation of vend switch 54 causes actuator 3 to remove a single medication canister 2 from container tray 12, causing medication canister 2 to roll down the incline into dispensing tray 4, as shown in FIG. 16c. The patient/user then removes the dispensed medication canister 2 from dispensing tray 4, as shown by FIG. 16d. Additionally, actuator 3 engages with the next medication canister 2 in the sequence of medication canisters 2 of array 9, locking the next medication canister 2 in position for vending at the next prescribed dosing time, as shown by FIG. 16e. FIG. 16f shows a condition of dispenser 1 wherein all medication canisters 2 have been dispensed from array 9 and container tray 12 is empty, wherein an indicator light, in some embodiments, communicates to the user or caregiver that container tray 12 requires reloading with a new sequence of medication canisters 2.

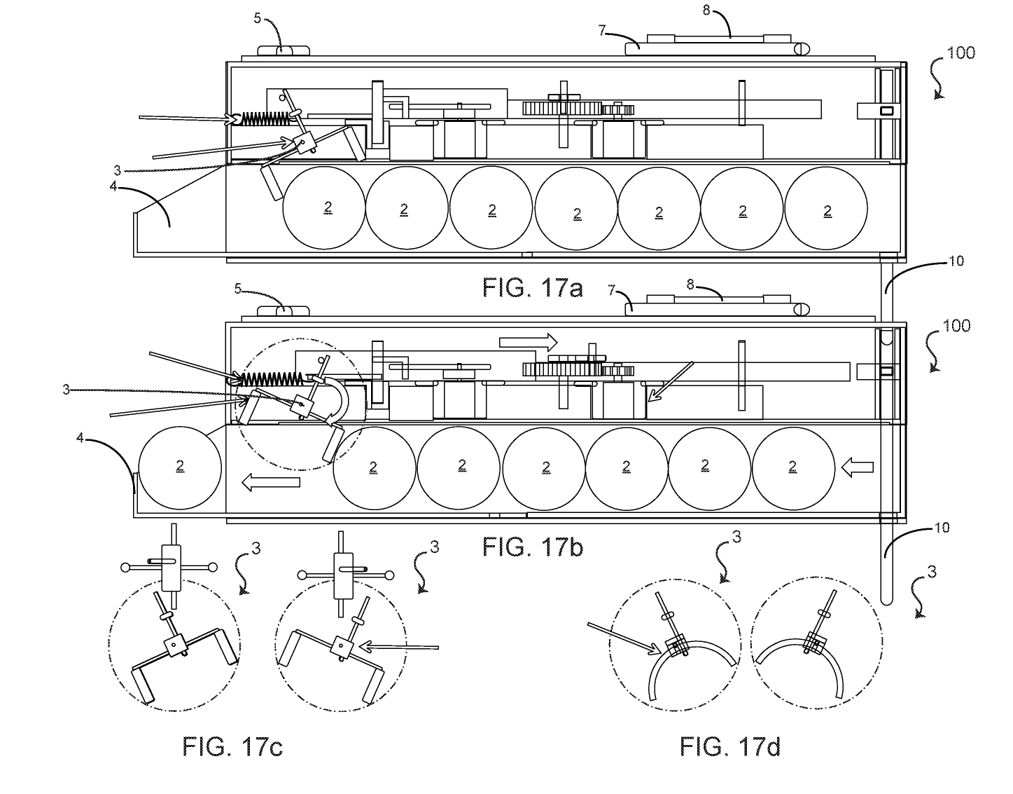

FIGS. 17a-d are detail side-cutaway views of an example actuator mechanism of a medication dispenser and monitoring system. This actuator mechanism is understood to be non-limiting; alternative actuator mechanisms known in the art are possible to engage medication canister 2, causing removal or medication canister 2 from canister tray 12. FIGS. 17a-d shows detail of one non-limiting example of an actuator 3 of dispenser 1. In this figure the send button has been pushed and the actuator begins to transition from the hold mode to the vend mode. FIG. 17a shows a first motor activated to extend an actuator arm movably coupled to a rocker. Two possible rocker shapes are shown in FIGS. 17a-d. The rocker is engaged with a medication canister 2. Extension of the actuator causes the rocker to rotate. moving medication canister 2 to a position wherein medication canister 2 becomes disengaged from the rocker of actuator 3 and rolls by gravity into dispensing tray 4. In some embodiments, a tensioned return spring rotates the rocker in the opposite direction, causing the rocker to engage the next medication canister 2 of array 9. Some embodiments of system 100 comprise a separate actuator mechanism for each row of array 9. Some embodiments of system 100 comprise a single actuator mechanism for all row of array 9, wherein the single actuator mechanism may move linearly to cause actuator 3 to engage with medication canisters 2 of any row comprising array 9. In some embodiments of system 100 comprising a single actuator mechanism, a selector slider (not shown) is coupled to the actuator mechanism. The selector slider comprises a linear track slidably coupled to the actuator mechanism, wherein an operator may operate the selector slider to move the actuator mechanism between rows of array 9 to engage medication canisters 2 for dispensing from each of the rows of array 9.

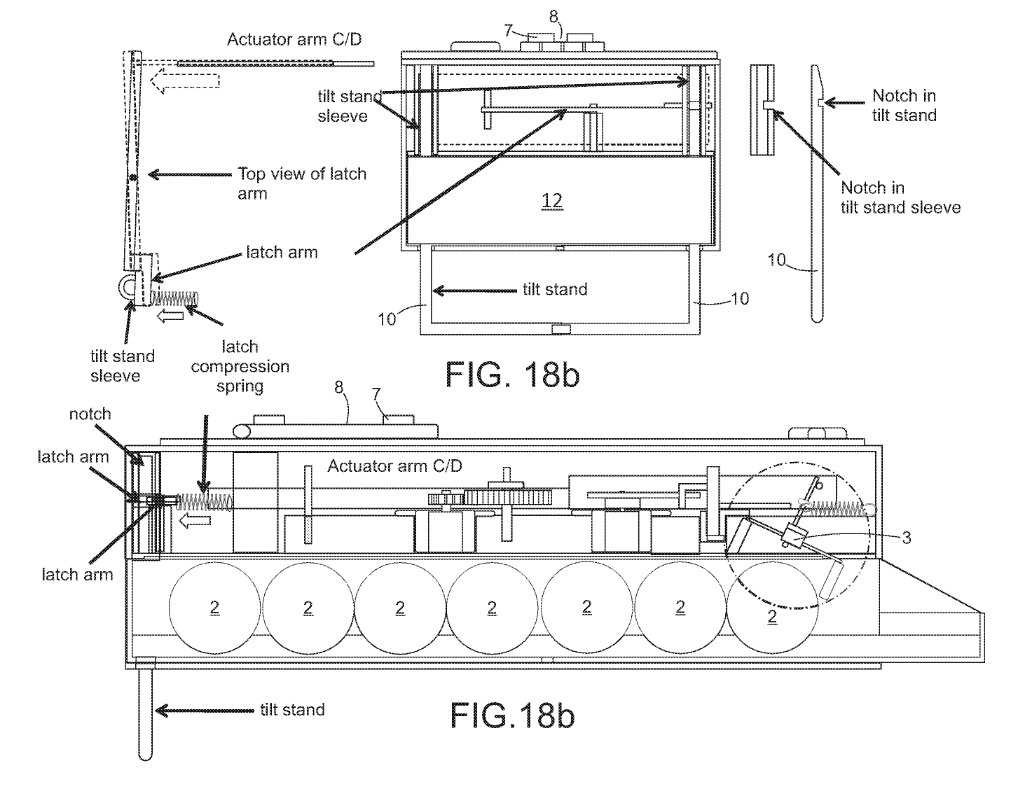

FIGS. 18a-b are side cutaway views of a dispenser of a medication dispenser and monitoring system having an internal latch. FIGS. 18a-b show internal latch 66 present in some embodiments of system 100 to deter tampering with dispenser 1 and theft of medications from inside dispenser 1. Internal latch 66 becomes locked, in some embodiments, upon insertion of tilt stand 10 into dispenser 1 whereupon an end of tilt stand 10 engages with internal latch 66. In some embodiments, internal latch 66 comprises a latch compression spring 67 that biases internal latch 66 into a locked position. An unlocking mechanism (not shown) opens internal latch into an unlocked position, compressing latch compression spring 67. Under a condition wherein the unlocking mechanism is released and tilt stand 10 is inserted into dispenser 1, internal latch re-engages into the locked position with the end of tilt stand 10, preventing removal of tilt stand 10 and locking container tray 12 in position for operation of system 100. shows how the tilt stand is locked into the pill dispenser by the latch located in the actuator assembly. Internal latch 66, in some embodiments is disposed at a rear end of dispenser 1.

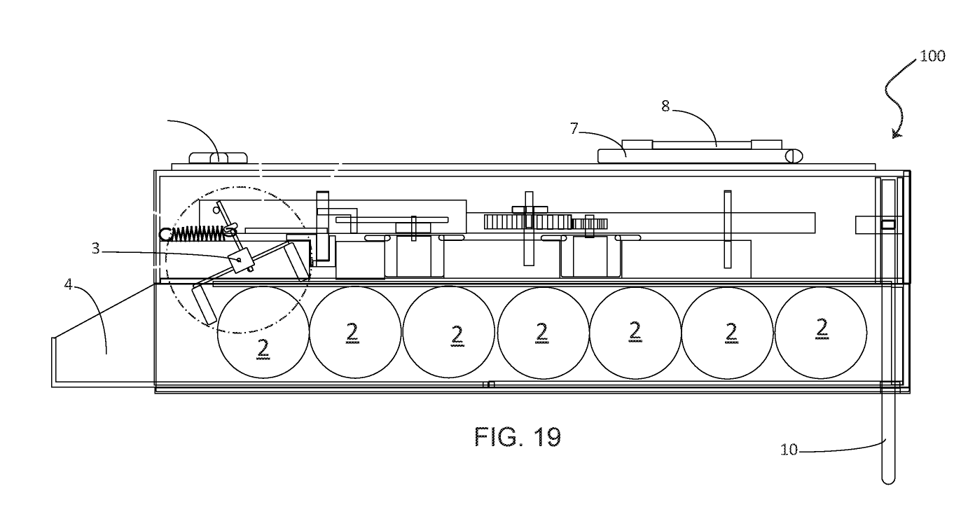

FIG. 19 is a top cutaway view of a dispenser of a medication dispenser and monitoring system. FIG. 19 shows the legs of tilt stand 10 tapered, wherein upon insertion of the legs into a tilt stand sleeve contained within dispenser 1, the taper of the leg engages with a feature of internal latch 66 to release internal latch 66. When the leg of tilt stand 10 disengages from internal latch 66, tilt stand 10 can be removed from dispenser 1, thereupon unlocking and releasing container tray 12 for removal from dispenser 1 for refill of medication canisters 2 and reloading of container tray 12.

FIG. 20 is a circuit block diagram of a medication dispenser and monitoring system. FIG. 20 schematically diagrams connections between the various electrical components of system 100. Power to energize system 100 is provided by a battery (not shown) of mobile computing device 8. Back-up power, in some embodiments, is provided from an AC line outlet via auxiliary power adaptor 11, which comprises an AC-DC converter. In the embodiment of system 100 shown by FIG. 20, and in some other embodiments, dispenser 1 comprises a first actuator motor and a second actuator motor, a vend switch, an emergency button, an opto-sensor, and an array of indicator lights. Dispenser 1 additionally comprises an actuator circuit board having any combination of a second microprocessor, a back-up time clock, a second memory, a tilt sensor, a motor driver electrically coupled to a first actuator motor or a second actuator motor, an LED driver, an electrical buffer coupled to vend switch 54, the opto switch, and emergency switch 6, and a direct current power management relay. Also shown by FIG. 20 are wireless couplings of additional elements to mobile computing device 8, including a Bluetooth coupling with a user-worn emergency button and a patient health monitor and a Wi-Fi coupling to an external video monitor, and a wireless cellular network modem.

In some embodiments, mobile computing device 8 is coupled through a USB connector to a DC power management circuit. The DC power management circuit distributes regulated voltages to the various circuit elements and mobile computing device 8. The DC power management circuit monitors incoming power from auxiliary power adaptor 1 and signals microprocessor 40 to switch over to converted line AC power, wherein the battery of mobile computing device 8 is depleted. In some embodiments, the buffer receives information from a circuit in pill dispenser 1 then stores and formats it for a data bus. Emergency switch 6 and vend switch 54, in some embodiments, are simple contact closures resulting in DC voltage change from supply to zero. A signal from an opto-switch, in some embodiments, is a DC voltage change from zero to supply if a phototransistor comprising the opto-switch turns off due to loss of a reflected IR light beam occurring when an actuator arm or rotor reaches a desired position. To conserve power and reduce heat, the opto-switch is sampled for about 10 ms every second and a buffer stores the sampled data, make a decision “present” or “not present” that is one or zero for data bus 118. The buffer also transfers data bidirectionally between the telephone modem, in some embodiments. LED drivers receive data from the data bus and respond by causing an LED disposed on housing 65 of dispenser 1 to become energized.

In some embodiments, microprocessor 40 receives and sends data over the data bus, periodically updates a real-time clock according to time data received over a cellular network and it must store and retrieve data from a nonvolatile memory. Embedded mobile computing device 8 transfers data between a variety of devices over the cellular network, as shown. A mini USB cable electrically and communicatively couples mobile computing device 8 with an actuator circuit board.

In some embodiments, red, yellow and green alert lights 55 are warning indicators chosen for high luminous output, only one is on at a time so power dissipation is not a concern. The red flashes at a rate optimized to attract attention. The yellow flashes at a slow rate and the green comes on steady for a longer time to alert the user that it is time to activate vend switch 54. LED drive waveforms are generated by an LED driver circuit. The LED driver circuit is designed to receive commands from the data bus and provides several choices of waveform for driving each LED. The red warning LED illuminates if the emergency switch 6 is activated and, in some embodiments, flashes until an “okay” signal is received on the incoming data stream. A motor driver drives a step motor with a prescribed waveform to move clockwise or counter clockwise until a position-sensing opto-switch signals that a desired position has been reached. The duration of this drive is typically about two seconds to minimize power consumption. In some embodiments, a tilt sensor is used to verify that pill dispenser 1 is in a normal operational tilt position. If the operational tilt position is incorrect, the tilt sensor sends an inhibit signal to microprocessor 40 blocking activation of vend switch 54 and, therefore, blocking initiation of an actuator motor driver sequence to vend medication canister 2. The operational tilt position of dispenser 1 is important because proper operation of pill dispenser 1 depends upon modest gravitational force to roll medication canister downward into dispensing tray 4.

FIG. 21 is a schematic representation of a medication dispenser and monitoring system. FIG. 21 shows system 100 comprising dispenser 1 communicatively coupled by communications link 35 (not shown) to multiple patient monitoring devices and the patient-worn emergency button through wireless Bluetooth connections. Dispenser 1 is also communicatively coupled to an external cellular network by a modem. System 100 exchanges signals through the external cellular network to deliver monitored clinical parameters, such as temperature or heartrate, for example, measured by the wireless patient monitoring device to a healthcare provider or caretaker located remote from the user. In some embodiments, communications link 35 additionally transmits voice, text, or video signals between the user and remote providers, either uni-directionally or bi-directionally. In some embodiments, system 100 is coupled to a wired telephone landline or DSL line for transmission of audio or video data.

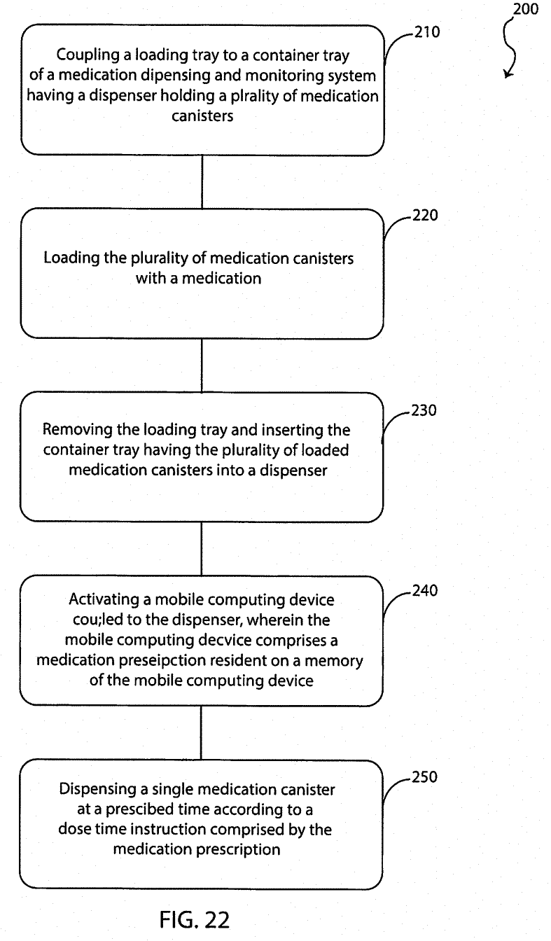

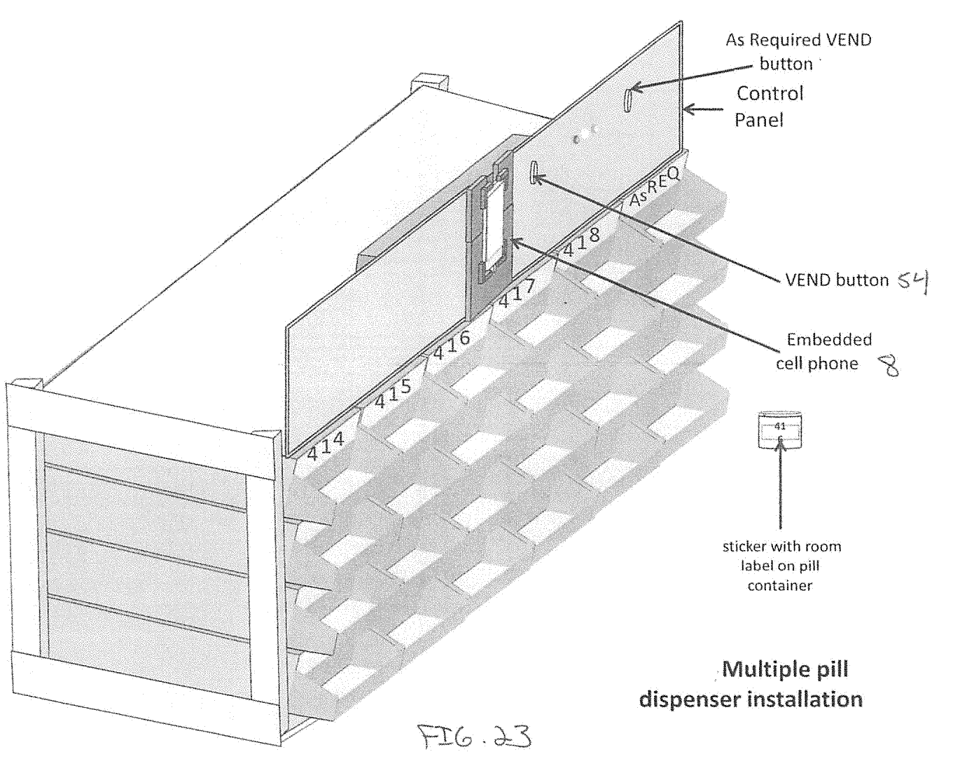

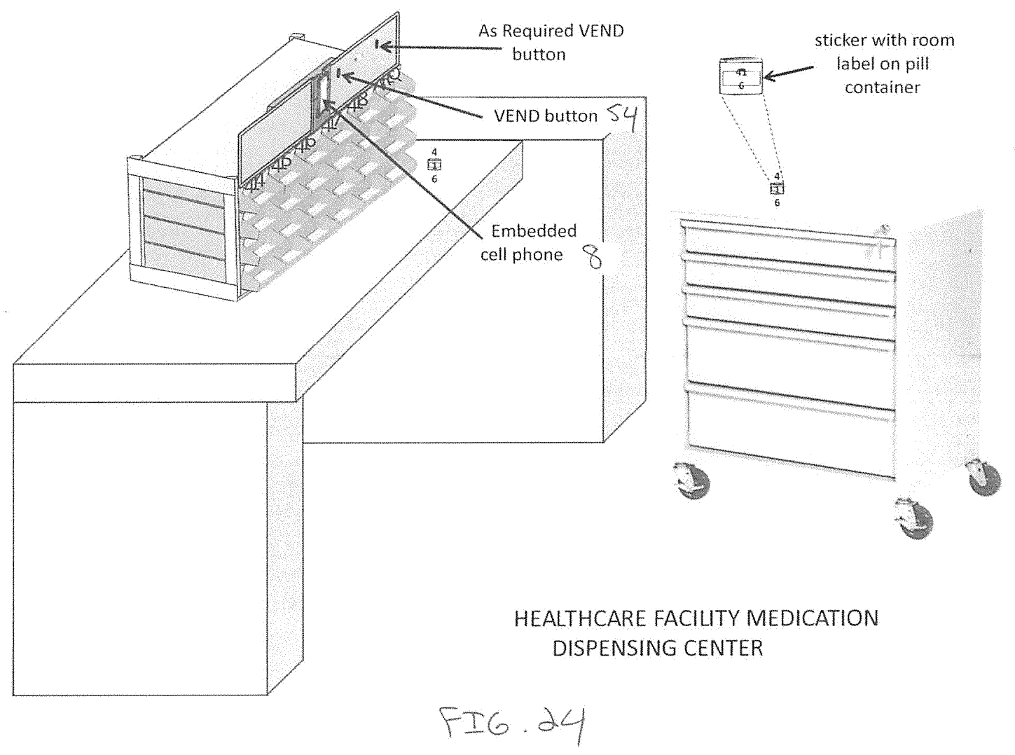

A variety of patients each requiring a different care regimen and medications in their homes can be served and monitored with two-way data sent over the cellular network or the Internet to exchange data with a hub, as shown in FIG. 22. The hub is a center for educational information, data collection, information disbursement, alert notifications and even 911 call transfers. In some embodiments, the hub is located in the healthcare provider's electronic medical record center. Data exchanged between the hub to the user's home may include video and voice as well as short data bursts for warnings and alerts. The data from the home to the hub may include voice or even video but primarily consists of short data bursts for compliance reporting, patient health status monitoring data and sometimes emergency help requests with activation of emergency switch 6.

It is important to minimize the amount of data that is sent out bound from the user's home to minimize the monthly data usage fees charged by the cellular service provider, and so that only a few phone numbers are required at hub. To accomplish this embedded mobile computing device 8 of system 100 will initiate a call to a second or a third numbers if it encounters a busy signal. Mobile computing device 8 will continue calling at random times until the hub answers and accepts it's short message typically a few seconds long. By monitoring peak cell phone activity the hub can determine if it needs to add more phone lines.

Within the patient's home dispenser 1 serves the patient in a number of ways: 1) It collects data from patient health monitor devices; 2) dispenses medication canisters on an approved schedule; 3) records when medication canisters 2 have been vended from dispenser 1; and 4) provides instruction and assistance to those responsible for error-free loading of pills in accordance with the approved schedule. At the hub, compliance and patient health monitor data with timestamps are collected for each patient as they are received. Algorithms resident on memory 41 of remote computing device 8 and the data center at the hub are used to monitor compliance and issue warnings, if needed. If an unacceptable condition is found, both the hub and remote computing device 8 generate an alert to the patient and also send out an alert to stakeholders via the Internet and/or the cellular network depending upon the defined protocol, in some embodiments.

The compliance and health monitor data collected by the hub is formatted for ease of use and distributed to the stakeholders and to remote computing device 8. In some embodiments, there may be a need to share data with hospitals, other healthcare providers or research facilities—this is done using the Internet. In the case of hospitals, if the patient is admitted the best situation is to have the patient bring pill dispenser 1 when the patient is admitted so in this case the hub now communicates with the hospital providing past records and current medication detail via an approved medication table. In the case of research facilities, the records accumulated by the hub may prove valuable in analyzing and modifying protocols for treating various conditions.

In some embodiments, a smart TV and related devices, such as ROKU, afford a Wi-Fi link to display information from embedded mobile computing device 8 coupled to dispenser 1 on the large screen of TV. The patient's television set may be used to see instructional video, still video of the approved medication dose timetable to assist in pill loading and other information that may be useful to the patient or healthcare providers.

In some embodiments, a physiologic measuring device may include any single device or combination of devices including a digital device for measuring blood pressure, a digital scale, a body temperature thermometer, a heartrate monitor, or the like. These devices transfer data to the pill dispenser 1. Many of these devices currently are available with a Bluetooth capability.

Some patients and users may take advantage of their existing Wi-Fi system to link to stakeholders and the hub using the Internet. The wireless modem and Internet cable modem are optional additions to the system.

In case of medical or other emergency the patient can summon 911 by activating emergency switch 6. This makes an emergency connection using the cellular network or Internet through the hub or directly to a 911 center.

The components defining any medication dispenser and monitoring system may be formed of any of many different types of materials or combinations thereof that can readily be formed into shaped objects provided that the components selected are consistent with the intended operation of a medication dispenser and monitoring system. For example, the components may be formed of: rubbers (synthetic and/or natural) and/or other like materials; glasses (such as fiberglass) carbon-fiber, aramid-fiber, any combination thereof, and/or other like materials; polymers such as thermoplastics (such as ABS, Fluoropolymers, Polyacetal, Polyamide; Polycarbonate, Polyethylene, Polysulfone, and/or the like), thermosets (such as Epoxy, Phenolic Resin, Polyimide, Polyurethane, Silicone, and/or the like), any combination thereof, and/or other like materials; composites and/or other like materials; metals, such as zinc, magnesium, titanium, copper, iron, steel, carbon steel, alloy steel, tool steel, stainless steel, aluminum, any combination thereof, and/or other like materials; alloys, such as aluminum alloy, titanium alloy, magnesium alloy, copper alloy, any combination thereof, and/or other like materials; any other suitable material; and/or any combination thereof.

Furthermore, the components defining any medication dispenser and monitoring system may be purchased pre-manufactured or manufactured separately and then assembled together. However, any or all of the components may be manufactured simultaneously and integrally joined with one another. Manufacture of these components separately or simultaneously may involve extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled with one another in any manner, such as with adhesive, a weld, a fastener (e.g. a bolt, a nut, a screw, a nail, a rivet, a pin, and/or the like), wiring, any combination thereof, and/or the like for example, depending on, among other considerations, the particular material forming the components. Other possible steps might include sand blasting, polishing, powder coating, zinc plating, anodizing, hard anodizing, and/or painting the components for example.

FIG. 21 is a flowchart showing a method of using a medication dispenser and monitoring system. FIG. 21 shows method 200 comprising a coupling step 210, a loading step 220, an uncoupling step 230, an activating step 230, and a dispensing step 240.

Coupling step 210, in some embodiments, comprises coupling a loading tray to a pill caddy of the medication dispensing and monitoring system having a dispenser holding a plurality of medication canisters. Coupling step 210 may be performed manually, or by an automated device by bringing the loading tray in contact with the pill caddy. In some embodiments, a surface feature of the loading tray removably engages with a corresponding surface feature of the pill caddy although this is not intended to be limiting. The loading tray comprises a row of at least two compartments and a shared compartment. Each of the at least two compartments, in some embodiments, is partially separated from the other compartments of the row, and each row completely separated from every other row. The loading tray has at least one row for each different medication to be loaded into each of the plurality of medication canisters.

Loading step 220 comprises loading the plurality of medication canisters with a medication. Loading step 220, in some embodiments, comprises multiple sub-steps which include filling a pill corral compartment with multiple pills of a single medication, pushing a single dose of pills from the pill corral compartment over a first ramp into a pill chute compartment, verifying visually that the proper number of pills corresponding to a single dose of the medication occupies the pill chute compartment, pushing the pills comprising the single dose over a second ramp and through a gate into a common fill port shared by a plurality of rows of the loading tray, opening a sliding cover allowing the pills comprising the single dose to fall into an open medication canister, moving the pill caddy to position a second medication canister beneath the sliding cover, and repeating the aforementioned sub-steps until all of the medication canisters held by the pill caddy are loaded, each with a single does comprising one or more medications. In some embodiments, the medication canisters are emptied from the pill caddy into a dispensing tray of a dispenser of the medication dispensing and compliance system and the pill caddy is filled with additional, empty open medication canisters. Loading of the additional medication canisters is performed, according to the aforementioned sub-steps, and loading step 220 continues until all of the medication canisters to be placed in the dispenser are loaded with medications.

Removing step 230, in some embodiments, comprises removing the loading tray and inserting the container tray having the plurality of loaded medication canisters into the dispenser. In some embodiments, the container tray is locked into the dispenser by a locking internal latch which engages a tilt bar inserted through the dispenser into a receiving sleeve of the internal latch. In some embodiments, removing step 230 comprises engaging an actuator with one of the medication canisters, locking all of the medication canisters in place within the container tray to hinder tampering with or theft of the medication canisters.

Activating step 230 comprises activating a mobile computing device coupled to the dispenser, wherein the mobile computing device comprises a medication prescription resident on a memory of the mobile computing device. In some embodiments, activating step 230 additionally comprises communicatively coupling an application resident on a memory of the mobile computing device with the actuator engaged with the one of the medication canisters.

Dispensing step 240 of method 200, in some embodiments, comprises dispensing a single medication canister at a prescribed time, according to a dose time instruction comprised by the medication prescription. In some embodiments, dispensing step 240 comprises viewing an alert, such as an illuminated LED alert externally located on a housing of the dispenser and activating a vend switch on the housing of the dispenser, causing the actuator to remove one of the medication containers from the container tray, wherein the removed medication container rolls out of the dispenser into a dispensing tray for retrieval by the user.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

What is claimed is:

1. A medication dispensing and monitoring system comprising:
A dispenser having
a container tray having a dispensing tray located proximate to a first end;
an actuator coupled to a sensor,
a plurality of medication canisters, wherein each medication canister is removably contained in the container tray,
an actuator operatively coupled to the container tray, wherein energizing the actuator causes a transfer of the medication canister from the container tray into the dispensing tray; and
a communications link having
a first microprocessor coupled to the actuator and the sensor,
a mobile computing device having
a second microprocessor,
a wireless link to a cellular network,
a display coupled to the second microprocessor,
a user interface coupled to the second microprocessor,
a memory coupled to the second microprocessor,
a prescribed dosing schedule residing on the memory, and
an application residing on the memory, wherein the actuator transfers the medication canister from the container tray into the dispensing tray at a prescribed time in response to a signal received from the microprocessor at a time according to the prescribed schedule.

2. The medication dispensing and monitoring system of claim 1, wherein the sensor is a pressure sensor.

3. The medication dispensing and monitoring system of claim 1, wherein the sensor is an optical sensor.

4. The medication dispensing and monitoring system of claim 1, further comprising a microphone and a speaker communicatively coupled to the second microprocessor.

5. The medication dispensing and monitoring system of claim 1, further comprising a camera communicatively coupled to the second microprocessor.

6. The medication dispensing and monitoring system of claim 1, wherein one of the plurality of medication canisters comprises
a unitary body with at least two sides; and
A lid mechanically coupled to the unitary body, wherein the lid is movable between a closed position and an open position with respect to the unitary body.

7. The medication dispensing and monitoring system of claim 1, wherein the communications link comprises a wireless coupling between the memory and a health monitoring system.

8. The medication dispensing and monitoring system of claim 1, further comprising an emergency button coupled to the communications link.

9. The medication dispensing and monitoring system of claim 1, wherein the communications link comprises a data upload to a healthcare provider, a family member, or an emergency service provider.

10. The medication dispensing and monitoring system of claim 1, further comprising a loading tray removably coupled to the container tray, having:
an array of compartments comprising
a pill corral,
a pill chute coupled to the pill corral by a first ramp,
a loading area coupled to the pill corral by a second ramp, and
a fill port coupled to the pill corral by a gate movable between a gate closed position and a gate open position;
a sliding cover slideably coupled to the loading area and interposed between the loading area and the fill port; and
the medication canister located beneath the sliding cover in the container tray.

11. The medication dispensing and monitoring system of claim 10, wherein the loading tray additionally comprises a magnifying viewer.

12. The medication dispensing and monitoring system of claim 1, further comprising
a camera communicatively coupled to the memory and the second microprocessor; and
a prescription container platform, wherein the camera
is activated to capture an image of a prescription label affixed to a medication container placed on the prescription container platform, and
transfer the image to the memory.

13. The medication dispensing and monitoring system of claim 12, wherein the image is a panoramic image.

14. The medication dispensing and motoring system claim 1, wherein the communications link comprises a receiving dock and a mobile computing device coupled to the receiving dock, wherein the mobile computing device is electrically coupled to the first microprocessor, and
comprises the second microprocessor and the memory.

15. The medication dispensing and monitoring system of claim 14, wherein the actuator is prevented from functioning in response to a signal from the first microprocessor under a condition wherein the mobile computing device becomes electrically uncoupled from the actuator.

16. A medication dispensing and monitoring system comprising:
a power source;
a dispenser having
a plurality of medication canisters secured within a locked container tray
a dispensing tray external to the locked container tray,
an actuator electrically coupled to the power source, and
a first microprocessor coupled to the actuator;
a mobile computing device electrically coupled to the power source, the mobile computing device having
a second microprocessor,
a memory with an application and medication schedule resident on the memory, wherein the power source energizes the actuator in response to a signal from the second microprocessor to transfer one of the plurality of medication canisters into the dispensing tray at a time indicated by the medication schedule;
wherein the mobile computing device is fixedly coupled to the dispenser and wherein removal of the mobile computing device enables the first microprocessor to cause activation of the actuator at the time indicated by the medication schedule.

17. The medication dispensing and compliance monitoring system of claim 16, wherein the first microprocessor causes a signal to be transmitted to a healthcare provider in response to a condition wherein the remote computing device becomes removed from the dispenser, or wherein the remote computing device becomes electrically uncoupled from the actuator.

18. A method of using a medication dispensing and monitoring system comprising:
coupling a loading tray to a container tray of the medication dispensing and monitoring system having a dispenser holding plurality of medication canisters;
loading the plurality of medication canisters with a medication;
removing the loading tray and inserting the container tray having the plurality of loaded medication canisters into a dispenser;
activating a mobile computing device coupled to the dispenser, wherein the mobile computing device comprises a medication prescription resident on a memory of the mobile computing device; and
dispensing a single medication canister at a prescribed time according to a dose time instruction comprised by the medication prescription.
